(19) United States
(12) Patent Application Publication
(10) Pub. No.: US 2023/0384279 A1
(43) Pub. Date: Nov. 30, 2023

(54) METHOD AND APPARATUS FOR OPEN MICRO-ENVIRONMENT PLANT HEALTH SENSING

(71) Applicant: PANASONIC FACTORY SOLUTIONS ASIA PACIFIC PTE.LTD., Singapore (SG)

(72) Inventors: Ishwariya VENKATESALU, Singapore (SG); Song Jin CHNG, Singapore (SG); Chin Huat Joel LIM, Singapore (SG); Cheng Yang Nicholas THAM, Singapore (SG); Mun Ji LOW, Singapore (SG)

(21) Appl. No.: 18/318,361

(22) Filed: May 16, 2023

Related U.S. Application Data
(60) Provisional application No. 63/347,164, filed on May 31, 2022.

(51) Int. Cl. G01N 33/00; A01G 7/02

(52) U.S. Cl. CPC G01N 33/0098; A01G 7/02

(57) ABSTRACT

An open micro-environment plant health sensing method includes placing a plurality of sensors in a configurable plant growing base for growing plants, collecting data representing one or more environmental parameters of the plants by the plurality of sensors continuously in real-time, communicating the collected data representing the one or more environmental parameters of the plants through a sensor network mediated by gateways, applying the data representing the one or more environmental parameters to a trained artificial intelligence (AI) engine for correlating micro-environment changes to the plants with one or more plant health parameters, and predicting the one or more plant health parameters for monitoring plant health by the trained AI engine.

### CROSS-REFERENCE TO RELATED APPLICATION

This application claims the benefit of Provisional Application No. 63/347,164, filed on May 31, 2022, the content of which is incorporated herein by reference in its entirety.

### TECHNICAL FIELD

The present disclosure relates to the field of farming and related agricultural and horticultural activities, and in particular relates to a method and an apparatus for open micro-environment plant health sensing to identify and monitor plant health through plant health parameters continuously in real-time without directly affecting plants.

### BACKGROUND

Indoor farming can be susceptible to systemic issues. For example, abiotic stress and biotic stress can negatively affect metabolomics of plants. However, it is impossible to measure all stress factors that may affect plants. Furthermore, plants take a long time to react to stress factors or other changes. It may also be difficult to relate plant health with any of these stress factors and how such factors may affect the plant health. In fact, by the time such relationships can be established, it would be too late to make any changes and prevent plant or plant losses from stress.

Moreover, sensors and devices capable of measuring plant parameters accurately and in real-time can be expensive, bulky and even cause additional stress under extended periods of monitoring. It can also be difficult to scale placement of the sensors and devices in the farm for monitoring the plant growth, especially for mass monitoring of the plants.

Therefore, a low-cost apparatus and method for real-time and continuous monitoring of plant health parameters are needed.

### SUMMARY

According to one aspect of the present disclosure, an open micro-environment plant health sensing method is provided. The method includes placing a plurality of sensors in a configurable plant growing base for monitoring plants grown on the base, collecting reference data representing one or more micro-environmental parameters of the plants by the plurality of sensors continuously in real-time, communicating the collected reference data through a sensor network mediated by gateways, applying the reference data representing the one or more environmental parameters to a trained artificial intelligence (AI) engine for correlating micro-environment changes to the plants with one or more plant health parameters, and predicting the one or more plant health parameters for monitoring plant health by the trained AI engine. The plurality of sensors are configured to measure the one or more micro-environmental parameters of the plants being monitored.

According to another aspect of the present disclosure, an apparatus for an open micro-environment plant health sensing is provided. The apparatus includes a configurable plant growing base for growing plants without directly affecting plants, a plurality of micro-environment sensors placed near the plants and configured to measure one or more micro-environmental parameters of a space near the plants in the configurable plant growing base continuously in real-time and a dashboard configured to display predicted plant health parameters.

### BRIEF DESCRIPTION OF THE DRAWINGS

A more particular description of the embodiments briefly described above will be rendered by reference to specific embodiments that are illustrated in the appended drawings. Understanding that these drawings depict only some embodiments and are not therefore to be considered to limit the scope, the embodiments will be described and explained with additional specificity and detail through the use of the drawings below.

FIG. 1 is a schematic diagram illustrating a relationship between metabolomics of plants and various abiotic stress and biotic stress factors;

FIG. 2A is a flowchart illustrating a training phase of a method for implementing an open micro-environment plant health sensing method according to some embodiments of the present disclosure; and FIG. 2B is a flowchart illustrating an implementation phase of the method for implementing the open micro-environment plant health sensing method according to some other embodiments of the present disclosure;

FIG. 3A is an image depicting the placement of a single micro-environment sensor near the plant according to some embodiments of the present disclosure; and FIG. 3B is another image depicting the placement of a plurality of micro-environment sensors near plants in an indoor farm or related environments according to some embodiments of the present disclosure;

FIG. 4 is a schematic diagram illustrating an implementation of an open micro-environment plant health sensing system according to some embodiments of the present disclosure;

FIG. 5 is another flowchart illustrating a process for implementing the open micro-environment plant health sensing method according to some embodiments of the present disclosure;

FIG. 6 is a chart illustrating comparisons among different experiment settings and corresponding experiment results for determining the optimal position for placing the micro-environment sensors according to some embodiments of the present disclosure; and

FIG. 7 is a schematic diagram showing placement of sensors for measuring environmental parameters and plant health parameters in addition to training and implementing real-time monitoring of plant health according to some embodiments of the present disclosure.

### DETAILED DESCRIPTION OF THE EMBODIMENTS

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

The phrase “in some embodiments” that appears in various places throughout this specification refers to the apparatus of this disclosure for implementing the micro-environment plant health sensing method in the detailed descriptions.

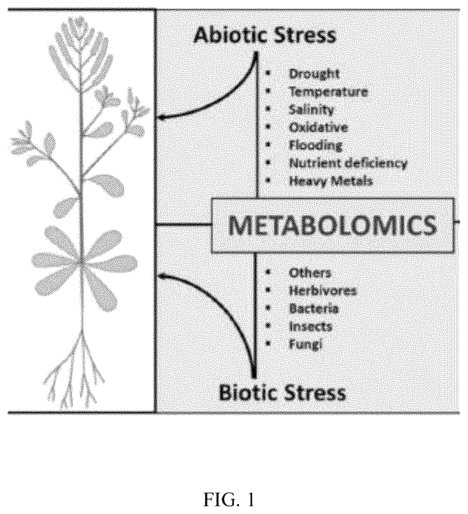

Plants may have health issues from biotic or abiotic factors. For example, plant stress, or infection by pests of disease through plant vitals may take place. There can be a variety of abiotic stress factors and biotic stress factors that may affect the metabolomics of the plants. FIG. 1 is a schematic diagram illustrating the relationship between metabolomics of plants and various abiotic stress and biotic stress factors. As shown in FIG. 1(a), the abiotic stress factors may include drought, temperature, salinity, oxidative, flooding, nutrient deficiency, heavy metals, etc. The biotic stress factors may include herbivores, bacteria, insects, fungi, and so forth.

It can be difficult to measure or track the health of plants. The device for measuring plant health, e.g., LI-COR LI-6800 portable photosynthesis system or other similar systems can be bulky and expensive. Such systems generally function by attaching or clamping an attachment over the leaf and measuring the total gaseous exchange to measure plant health parameters in a closed manner. Oftentimes, this type of device may be used only for a short amount of time as longer usage may induce stress to the plant from clamping of the leaf, and the staff who performs the measurement usually operates a single device for measurement, which may hinder the scalability for mass deployment at various plant base sections. As a result, many plant health issues cannot be identified in a speedy manner, especially in the context of a large farm or related agricultural or horticultural contexts.

Moreover, the plant stress or plant infections may be non-apparent and asymptomatic at the beginning. Indoor farms are particularly susceptible to systematic threats to plant health via biotic or abiotic factors (e.g., infestation, root issues, etc.) or system failures (e.g. damaged lights or nutrient feeding systems) which can speedily spread through or widely affect the indoor environment and plant population. For example, if the growing lights in the farm was damaged and light intensity reduced for an extended period, photosynthetic rate of the plants may reduce below ideal growth conditions. However, the negative impact on the plant growth may not be visible until the plant shows symptoms of light stress. Alternative methods to monitor plant health through the capture and analysis of plant images are not sufficient as well. Visual changes to plants due to stress, for example yellowing of leaves, would take up to a few days or longer to occur, by which the damage to the plants might be irreversible. When the consequences can be visible, the best timing for adjusting the environment would have passed.

The present disclosure provides a method and an apparatus for implementing open micro-environment-based plant health sensing. The method can be implemented in indoor farming using artificial intelligence (AI) to achieve real-time and constant monitoring of plant health parameters for quickly identifying negative plant stresses or infections by pests. The indoor farming may be an open micro-environment, and measurements can be taken from near-plant surroundings of the open micro-environment, By relying on measurements taken from near-plant surroundings without directly clamping or affecting the leaf, the open micro-environment, as opposed to obtaining closed measurement of plant health parameters in a closed environment of a portable photosynthesis system is made possible without directly affecting the plant. A system implementing the open micro-environment-based plant health sensing method may be capable of detecting the health of plants continuously and in real-time to enable rapid identification and treatment of plant stresses.

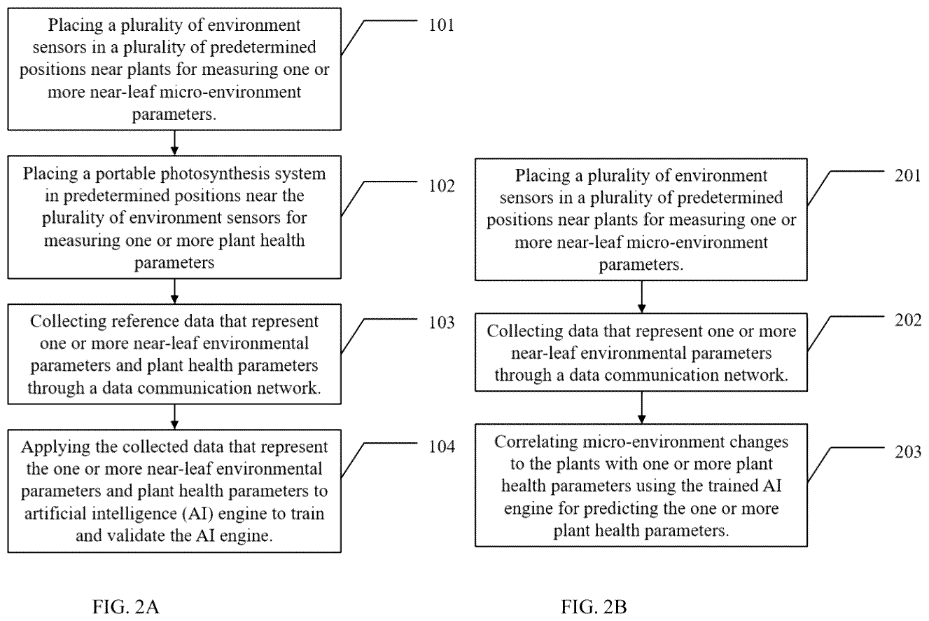

According to the embodiments of the present disclosure, the method for open micro-environment-based plant health sensing can be implemented in farm or related agricultural or horticultural context. The system may use environmental sensors to measure micro-environmental parameters at ideal locations near plants, and a trained artificial intelligence (AI) engine fed with the data collected from these sensors to link the environmental changes to the plant with the plant health thereby monitoring plant health continuously in real-time. This AI engine may be trained beforehand with reference to photosynthesis system, for example, the LI-COR LI-6800 portable photosynthesis system, or other similar systems, etc. An environmental parameter refers to a parameter that represents a condition of an environment. The conditions of the environment depend on a variety of environmental parameters. The environmental parameters may include but are not limited to relative humidity (RH), temperature (T), and carbon dioxide (CO₂). The plant health parameters may include but not limited to photosynthetic rate (A) and stomatal conductance (SC). Accordingly, the health of plants can be monitored within the farm at any given time. FIG. 2A is a flowchart illustrating a training phase of a method for implementing an open micro-environment plant health sensing method according to some embodiments of the present disclosure; and FIG. 2B is a flowchart illustrating an implementation phase of the method for implementing the open micro-environment plant health sensing method according to some other embodiments of the present disclosure. As shown in FIG. 2A, the method for open micro-environment plant health sensing may be implemented as follows, starting with the training phase.

Step 101: Placing a plurality of environment sensors in a plurality of predetermined positions near plants for measuring one or more near-leaf micro-environment parameters.

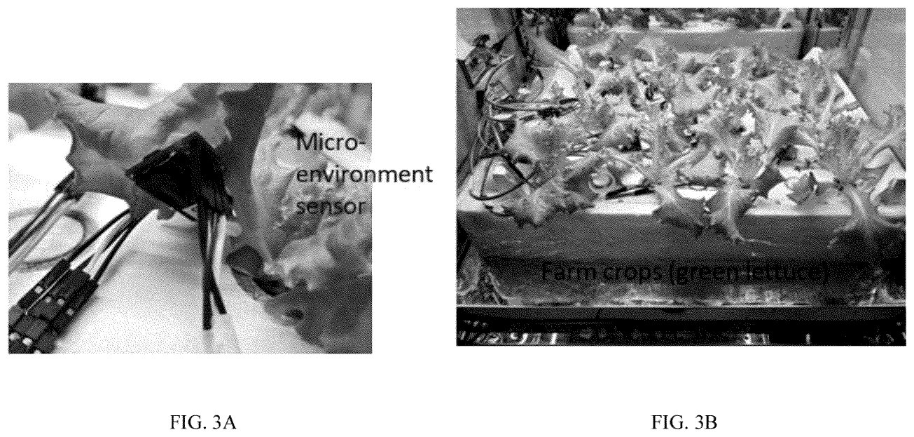

In step 101, a plurality of micro-environment sensors may be placed in a plurality of predetermined positions near plants for measuring near-leaf or on-leaf micro-environmental parameters. The predetermined positions may be in ideal positions near the plants for measuring near-leaf micro-environmental parameters without affecting the plants. FIG. 3A depicts the placement of a single micro-environment sensor near the plant according to some embodiments of the present disclosure. As shown in FIG. 3A, a micro-environment sensor is placed under a leaf to measure a corresponding near-leaf environmental parameter. FIG. 3B is another image depicting the placement of a plurality of micro-environment sensors near plants in an indoor farm or related agricultural or horticultural contexts, according to some embodiments of the present disclosure. As shown in FIG. 3B, a plurality of micro-environment sensors may be placed under each of the leaves in one of the compartments of an indoor farm. The plurality of micro-environment sensors may also operate as a network. These sensors collect data that represent one or more near-leaf environmental parameters.

According to the embodiments of the present disclosure, the near-leaf micro-environmental parameters may include but are not limited to relative humidity (RH), temperature (T), and carbon dioxide (CO₂).

Step 102: Placing a portable photosynthesis system in a predetermined position near the plurality of environment sensors for measuring one or more plant health parameters.

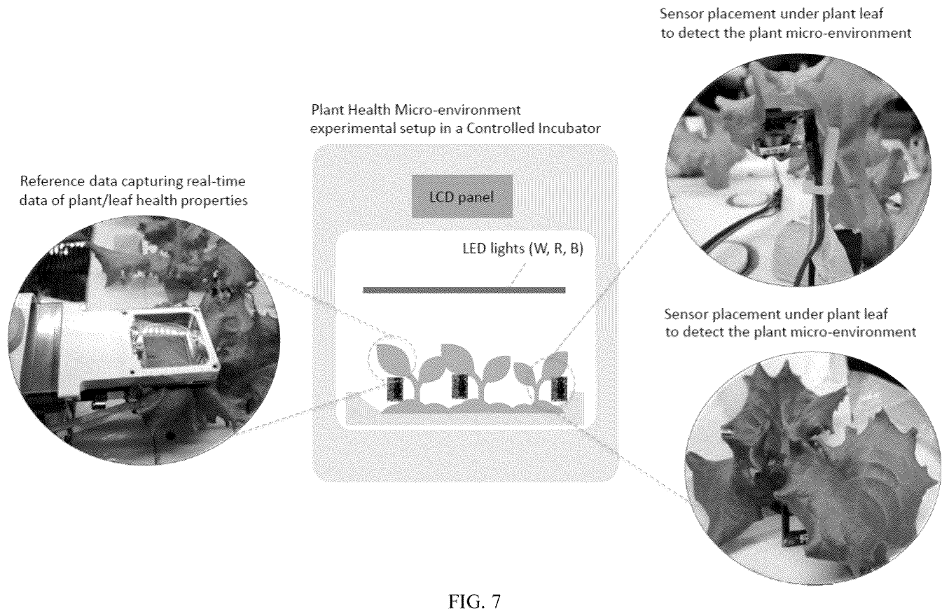

In step 102, a portable photosynthetic system may be placed in predetermined positions near plants for measuring plant health parameters. The predetermined positions may be near the ideal positions for measuring near-leaf micro-environmental parameters. FIG. 7 is a schematic diagram showing placement of sensors for measuring environmental parameters and plant health parameters in addition to training and implementing real-time monitoring of plant health according to some embodiments of the present disclosure. As shown in FIG. 7 (left image), the portable photosynthetic system may be clamped onto the leaf, and plant health parameters can be measured directly.

According to the embodiments of the present disclosure, the plant health parameters may include but are not limited to photosynthetic rate (A) and stomatal conductance (SC).

Step 103: Collecting reference data that represent one or more near-leaf environmental parameters and plant health parameters through a data communication network.

In step 103, after the plurality of micro-environment sensors and the portable photosynthetic system are placed at different positions in the indoor farm or related agricultural or horticultural contexts, the sensors and system will start to collect reference data that represent one or more near-leaf environmental parameters and plant health parameters respectively. In some embodiments, one or more near-leaf environmental parameters and plant health parameters may be collected through a data communication network, for example, through a sensor network mediated by gateways. After the reference data that represent one or more near-leaf environmental parameters and plant health parameters are collected, the reference data can be used for training a machine learning model.

Step 104: Applying the collected reference data that represent the one or more near-leaf environmental parameters and plant health parameters to artificial intelligence (AI) engine to train the AI engine.

In step 104, after reference data represent the one or more environment parameters are collected through a data communication network, the data may be then applied to train a developed AI prediction engine. The trained AI engine is then capable of predicting the photosynthetic rate and stomatal conductance of the plants, which are important for determining the health of the plants.

The AI approach may be enabled through Machine Learning, a subset of AI. A Machine Learning model may employ computational methods to learn through the training data to recognize hidden patterns between input and target parameters. The model may then be used to predict target variables, for example, the plant health parameters. The Machine Learning approach is adapted to predict plant health parameters from input sensed micro-environment variables of plant (e.g., RH, T and CO₂).

According to the embodiments of the present disclosure, training the AI engine includes validation of the predictions made of the plant health parameters to ensure high accuracy and precision of the predicted plant health parameters. Validation may be done via the use of data that represent the one or more near-leaf environmental parameters and plant health parameters previously unused and unseen from the training of the AI engine.

After training is completed, the open micro-environment plant health sensing method can be implemented as follows.

Step 201: Placing a plurality of environment sensors in a plurality of predetermined positions near plants for measuring one or more near-leaf micro-environment parameters.

Step 201 is similar to step 101. In step 201, a plurality of micro-environment sensors may be placed in a plurality of predetermined positions near plants for measuring near-leaf or on-leaf micro-environmental parameters. The predetermined positions may be in ideal positions near the plants for measuring near-leaf micro-environmental parameters.

According to the embodiments of the present disclosure, the near-leaf micro-environmental parameters may include but are not limited to relative humidity (RH), temperature (T), and carbon dioxide (CO₂).

Step 202: Collecting data that represent the one or more near-leaf environmental parameters through a data communication network.

Step 202 is similar to step 103. In Step 202, after the plurality of micro-environment sensors are placed at different positions in the indoor farm or related agricultural or horticultural contexts, the sensors and system may start to collect data that represent one or more near-leaf environmental parameters. In some embodiments, one or more near-leaf environmental parameters may be collected through a data communication network, for example, through a sensor network mediated by gateways. After the data that represent one or more near-leaf environmental parameters are collected, the data can be used to correlate micro-environment changes with one or more plant health parameters.

Step 203: Correlating micro-environment changes to the plants with one or more plant health parameters using the trained AI engine for predicting the one or more plant health parameters.

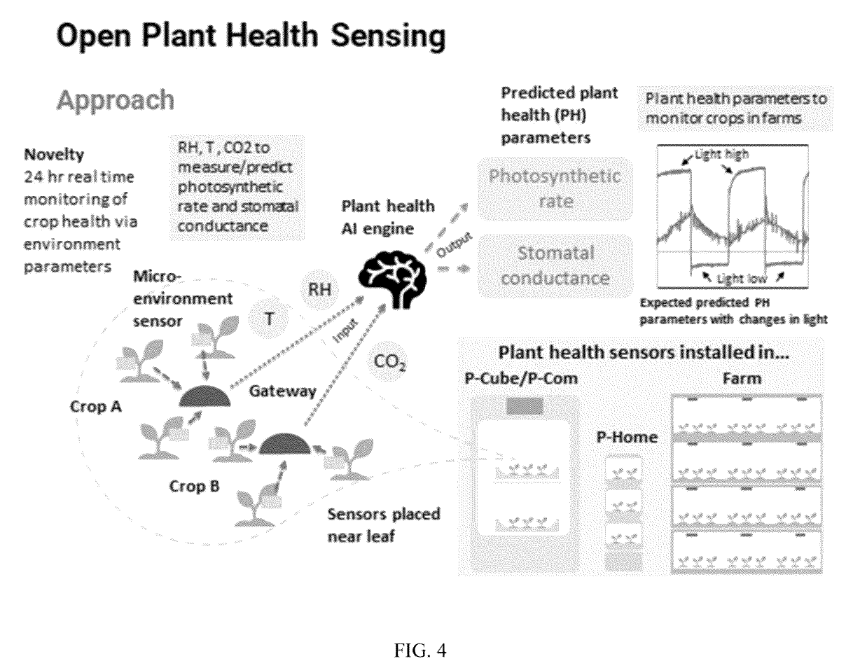

In step 203, the trained AI engine can be used to monitor the plant health. FIG. 4 is a schematic diagram illustrating implementation of a micro-environment plant health sensing system according to some embodiments of the present disclosure. As shown in FIG. 4, the trained plant health AI engine can output predicted plant health parameters. The predicted health parameters can be further used for monitoring plants in farms. The one or more plant health parameters may include, for example, photosynthetic rate (A) and stomatal conductance (SC). Micro-environment changes may then be correlated to plant health parameters such as PR and SC, for monitoring the plant health. As shown in FIG. 4, in one instance, the predicted plant health parameters may change significantly with light due to impact on PR.

In some embodiments, the plant health sensors may also be installed in an incubator with controllable environmental parameters. In some other embodiments, the plant health sensors may also be placed in indoor or outdoor farms, or related agricultural or horticultural contexts.

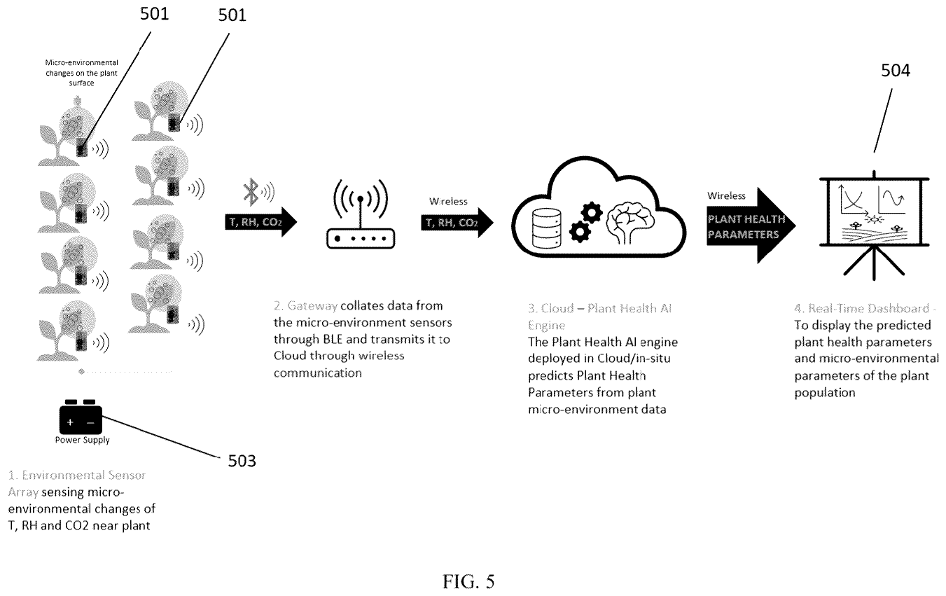

FIG. 5 is a flowchart illustrating a process for implementing an open micro-environment plant health sensing method according to some embodiments of the present disclosure. According to some embodiments of the present disclosure, as shown in FIG. 5, by placing a plurality of micro-environment sensors 501 near the plants, the plant health parameters can be measured in an open micro-environment 502. A power supply 503 may supply power to the plurality of micro-environment sensors 501. The open micro-environment 502 is accompanied by the open-loop feedback fashion thereby avoiding the traditional way to enclose the plants for measuring plant health parameters using portable photosynthesis systems. In this way, the placement of the micro-environment sensors may also be employed and rearranged easily.

According to the embodiments of the present disclosure, the health of the plants can be monitored continuously. For example, as shown in FIG. 5, micro-environmental changes of the one or more environmental parameters (i.e., T, RH, and CO₂) on the plant surface can be detected by the environmental sensor array. The data representing the one or more environmental parameters may be collected by the micro-environment sensors through a communication protocol continuously in real-time, e.g., Bluetooth Low Energy (BLE), and transmitted to the cloud through wireless connection e.g. Wi-Fi. In some embodiments, BLE based communication can facilitate low power consumption between sensor array and Gateway and transmit data in a time sequence fashion. Wi-Fi based communication may have the flexibility and power to transmit data to a long-distance range. HTTP/MQTT protocol may provide a lightweight publish-subscribing network transporting messages. The plant health AI engine may be located in the cloud or in-situ such that a visualization form for the plant health parameters may be displayed for evaluating the plant health. Alternatively, the AI engine may also be located in a local computer.

The AI engine may then generate predictions for plant health. In some embodiments, the AI engine can provide predicted health parameters for establishing a relationship between changes to the environmental parameters and the plant health parameters. For example, changes to the micro-environmental parameters may be associated with the photosynthetic rate such that the photosynthetic rate may be predicted to demonstrate plant health.

According to the embodiments of the present disclosure, as shown in FIG. 5, a real-time dashboard 504 may display the predicted plant health parameters along with real-time monitoring of the plant health in the micro-environment. The dashboard 504 may update the monitored plant health parameter in a predetermined duration, for example, from every two seconds to every 30 minutes, etc. In some embodiments, the dashboard 504 can be updated with predicted plant health parameters and real-time updates of plant environment data pursuant to other customized insights.

Plants tend to react to the environment over the time. The leaves of the plants breathe, photosynthesize, and transpire water, which may in turn affect temperature (T), relative humidity (RH), carbon dioxide (CO₂) or other environment parameters in the surrounding area, or micro-environment. As the plant breathes or grows, CO₂ and water vapor in the micro-environment may vary. Nonetheless, when the distance from the plant is far, the environmental parameters measured in that distant spot may not reflect any changes the plant undergoes. For example, the further the distance between the sensor and the plant-to-be-measured, the less likely any changes to the environment detected by the sensor can be attributed to changes in the plant's micro-environment. For example, the RH measured in the ideal near-plant sensing location may change from 0.5% to 1%, which can be directly related to changes in the plant's micro-environment, while a similar change measured at a distance 1 m away from the plant-to-be-measured cannot be directly related to changes in the plant's micro-environment.

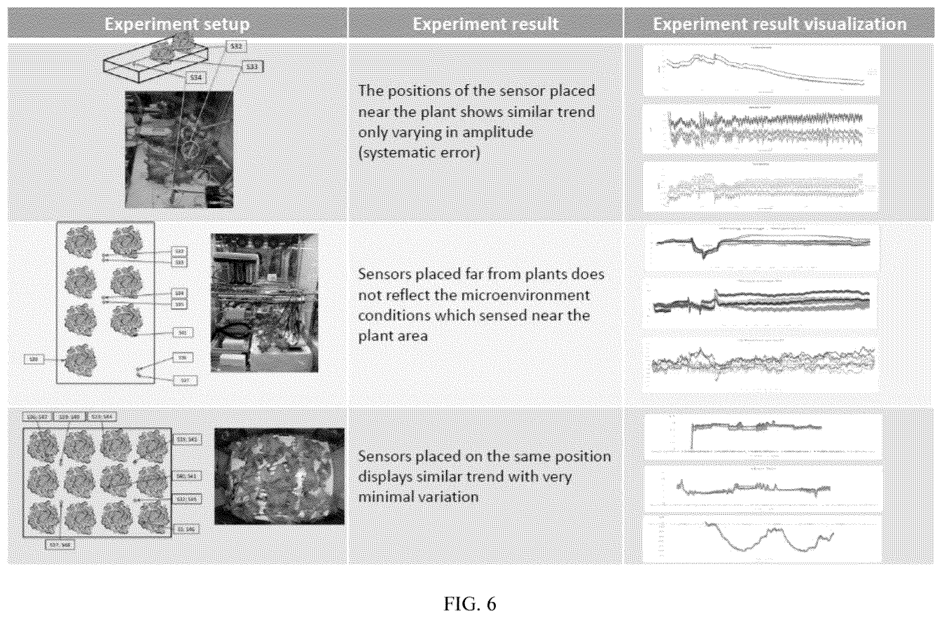

According to some embodiments of the present disclosure, a series of experiments may be conducted with different locations to determine the optimal location for placing the micro-environment sensors 501. The reference data collected from the series of experiments may be used to determine the optimal location for placing the environment sensors 501. In some embodiments, the reference data may include data representing both the one or more micro-environmental parameters of the plants. The reference data may be collected by the plurality of sensors 501. The optimal positions of placing the sensors, either close to the plant, or distant from the plant, may be determined based on the experiment results. FIG. 6 is a chart illustrating comparisons among different experiment settings and corresponding experiment results for determining the optimal position for placing the micro-environment sensors 501 according to some embodiments of the present disclosure. For example, as shown in the chart provided in FIG. 6, when the positions of the micro-environment sensors 501 are close to the plant-to-be measured, the predicted health parameters show a similar trend in their curves and only vary in amplitude considered to be a systematic error. On the other hand, when the positions of the micro-environment sensors 501 are distant from the plant-to-be-measured, the predicted health parameters may not reflect the microenvironment conditions representing the area close to the plant-to-be-measured. Further, the visualized experiment result shows consistent results when the positions of the sensors are the same with respect to the different leaf of the plants. That is, the experiment results when sensors are placed on the same position display a similar trend with minimal variation. In addition, when setting up the comparison experiments, the differences between the experimental results corresponding to a close position and the experiment results corresponding to a distant position may be captured. If the differences are within a predetermined range, then the sensors may be then placed under the leaves structurally.

Accordingly, the reference data representing both the one or more micro environmental parameters and plant health parameters are collected by the plurality of sensors and the portable photosynthesis system placed at the optimal positions. After data collection, the reference data may be communicated through the sensor network mediated by gateways and may be applied to train and validate the AI engine. After validation, the trained AI engine may be ready for taking plant health parameters for monitoring the plant health.

The Machine Learning model is developed per problem requirement and is trained through reference data collated through plant health experiments. The model validation process can be then employed to cross-validate the predicted results with real-world data.

According to some embodiments of the present disclosure, the plants-to-be-measured for reference data may be determined from all the plants in the configurable plant growing base. The reference data representing the one or more micro environmental parameters and plant health parameters may be collected for the plants-to-be-measured and then used for training the AI engine.

FIG. 7 is a schematic diagram showing the placement of sensors for measuring environmental parameters and implementing real-time monitoring of plant health according to some embodiments of the present disclosure. In some embodiments, as shown in FIG. 7, reference data is collected directly from the plant-to-be-measured. Micro-environment data of the plants may be collected for the same plant-to-be-measured, and then applied to the AI engine training process. The growth data of the plants may include the photosynthetic rate of the plants. Under different environment conditions, the photosynthetic rates may be different and hence, the experiments may be conducted at different environment parameters for data collection. After training, the AI engine may be used for predicting plant health.

Therefore, with a combination of open micro-environment data collection methodology, wireless and self-powered set-up as well as AI plant health parameter prediction engine may be implemented to achieve real-time and constant monitoring of plant health parameters. Multiple small environment sensors may be placed in ideal positions close to the plant to measure near leaf micro-environment parameters (e.g., RH, T, CO₂). After data collection, the reference data may be communicated through the sensor network mediated by gateways and may be applied to train and validate the AI engine. The trained AI engine can then be used to correlate micro-environment data to predict one or more plant health parameters. Micro-environment changes can then be correlated to plant health parameters such as photosynthetic rate (A) and stomatal conductance (SC), for monitoring plant health. This system is designed to be wireless and self-powered, for wide scale placement throughout a farm, so that any changes in plant parameters can be monitored continuously in real-time for tracking the health of plants. Thus, detecting the plant stresses and disease at an early stage can be implemented, through sensing the changes in plant micro-environment without directly affecting the plants.

According to the embodiments of the present disclosure, an apparatus for implementing open micro-environment plant health sensing may also be provided. The apparatus can implement the above methods to track plant health in an open micro-environment. The apparatus may include a configurable plant growing base 502 for growing plants, and a plurality of micro-environment sensors 501 configured to measure one or more environmental parameters of the plant micro-environment 502 without affecting plants. The apparatus may further include a dashboard 504. The dashboard 504 may be a physical or a web-based dashboard for displaying predicted plant health parameters.

The above description of the disclosed embodiments of the present disclosure can enable those skilled in the art to implement or use the present disclosure. Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. An open micro-environment plant health sensing method, comprising:
placing a plurality of sensors in a configurable plant growing base for growing plants for monitoring the plants, the plurality of sensors being configured to measure one or more micro-environmental parameters of the plants being monitored;
collecting, by the plurality of sensors, reference data representing the one or more environmental parameters of the plants continuously in real-time;
communicating the collected reference data through a sensor network mediated by gateways;
applying the reference data representing the one or more micro-environmental parameters to a trained artificial intelligence (AI) engine for correlating micro-environment changes to the plants with one or more plant health parameters; and
predicting, by the trained AI engine, the one or more plant health parameters for monitoring plant health.

2. The method according to claim 1, further comprising an AI training process, including:
placing a portable photosynthesis system in a predetermined position near the plurality of environmental sensors for measuring one or more plant health parameters;
collecting, by the plurality of sensors and portable photosynthesis system, reference data representing the micro-environmental parameters and plant health parameters of the plants;
communicating the reference data representing the one or more micro-environmental parameters and plant health parameters of the plants through the sensor network mediated by gateways; and
applying the reference data representing the one or more environmental parameters and plant health parameters of the plants and plant health parameters of the plants to train and validate the AI engine.

3. The method according to claim 2, wherein validation of the AI engine comprises:
using the reference data representing the one or more near-leaf environmental parameters and plant health parameters unused and unseen from the training of the AI engine.

4. The method according to claim 1, wherein the one or more micro-environmental parameters comprise relative humidity (RH), temperature (T), and carbon dioxide (CO₂) of the plants.

5. The method according to claim 1, wherein the one or more plant health parameters comprise photosynthetic rate (A) and stomatal conductance (SC).

6. The method according to claim 1, wherein the plurality of sensors comprises one or more of relative humidity (RH) sensor, carbon dioxide (CO₂) sensor, and temperature (T) sensor.

7. The method according to claim 1, further comprising:
determining a number of plants in the configurable plant growing base to be measured for the reference data;
collecting the reference data from sensors corresponding to the number of plants; and
training the AI engine using the reference data.

8. The method according to claim 1, further comprising:
determining, based on differences between the one or more health parameters corresponding to a distant sensor position with respect to a plant, and the one or more health parameters corresponding to a close sensor position with respect to the plant, optimal positions for placing the plurality of sensors with respect to the plants.

9. The method according to claim 1, further comprising:
displaying the predicted plant health parameters on a dashboard.

10. The method according to claim 1, wherein the dashboard updates the predicted plant health parameters based on a preset duration of time.

11. An apparatus for an open micro-environment plant health sensing, comprising:
a configurable plant growing base for growing plants;
a plurality of micro-environment sensors placed near the plants and configured to measure one or more environmental parameters of a space near the plants in the configurable plant growing base and
a dashboard configured to display predicted plant health parameters.

12. The apparatus according to claim 11, wherein the plurality of sensors comprises one or more of relative humidity (RH) sensor, carbon dioxide (CO₂) sensor, and temperature (T) sensor.

13. The apparatus according to claim 11, wherein the one or more plant health parameters comprise photosynthetic rate (A) and stomatal conductance (SC).
